(19) United States
(12) Patent Application Publication
(10) Pub. No.: US 2020/0005458 A1
(43) Pub. Date: Jan. 2, 2020

(54) DYNAMIC IMAGE PROCESSING APPARATUS AND RECORDING MEDIUM

(71) Applicant: KONICA MINOLTA, INC., Tokyo (JP)

(72) Inventor: Noritsugu MATSUTANI, Tokyo (JP)

(73) Assignee: KONICA MINOLTA, INC., Tokyo (JP)

(21) Appl. No.: 16/420,596

(22) Filed: May 23, 2019

(30) Foreign Application Priority Data
Jun. 28, 2018 (JP) 2018-122598

(51) Int. Cl. G06T 7/00; G06T 7/70; G06T 7/20; G06T 11/60; G16H 30/40; A61B 6/00

(52) U.S. Cl. CPC G06T 7/0014; G06T 7/70; G06T 7/20; G06T 11/60; G06T 2207/30061; A61B 6/5217; A61B 6/466; G06T 2207/10116; G16H 30/40

(57) ABSTRACT

A dynamic image processing apparatus includes a hardware processor that detects an abnormal site of an imaging object from a radiographically taken dynamic image, and identifies a three-dimensional position of the abnormal site from a positional relationship between the abnormal site of the imaging object detected from one dynamic image and the abnormal site of the imaging object detected from another dynamic image when the abnormal site is detected from a plurality of dynamic images radiographically taken in a plurality of directions.

### BACKGROUND

### Technological Field

The present invention relates to a dynamic image processing apparatus and a recording medium.

### Description of the Related Art

Conventionally, dynamic images obtained by radiation-imaging the dynamics of an imaging object having a cycle have been used for diagnosis. Through dynamic images, the dynamics of the imaging object that cannot have been grasped through still images can be displayed and analyzed.

For example, Japanese Patent Laid-open No. 9-187448 describes an X-ray image diagnostic apparatus that can obtain measured values in consideration of variation in the depth of a target object by calculating the local geometric magnification ratio at each position of the target object using X-ray images taken in multiple directions, in quantitative analysis of blood vessels and cardiac functions.

Unfortunately, the X-ray image diagnostic apparatus described in Japanese Patent Laid-open No. 9-187448 is an apparatus for quantitatively grasping the degree of an abnormal site, assuming that the position of the abnormal site is identified, and cannot perform a series of processes including detection of the abnormal site to identification of the three-dimensional position of the abnormal site.

### SUMMARY

The present invention has been made in view of the above problem, and has an object to provide a dynamic image processing apparatus and a recording medium that can easily perform the series of processes including detection of the abnormal site to identification of the three-dimensional position of the abnormal site.

To achieve at least one of the abovementioned objects, according to a first aspect of the present invention, a dynamic image processing apparatus reflecting one aspect of the present invention comprises

a hardware processor that detects an abnormal site of an imaging object from a radiographically taken dynamic image, and identifies a three-dimensional position of the abnormal site from a positional relationship between the abnormal site of the imaging object detected from one dynamic image and the abnormal site of the imaging object detected from another dynamic image when the abnormal site is detected from a plurality of dynamic images radiographically taken in a plurality of directions.

According to a second aspect of the present invention, a recording medium reflecting one aspect of the present invention comprises

a computer-readable recording medium storing a program for causing a computer to function as

a hardware processor that detects an abnormal site of an imaging object from a radiographically taken dynamic image, and identifies a three-dimensional position of the abnormal site from a positional relationship between the abnormal site of the imaging object detected from one dynamic image and the abnormal site of the imaging object detected from another dynamic image when the abnormal site is detected from a plurality of dynamic images radiographically taken in a plurality of directions.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 shows the entire configuration of a dynamic image processing system according to an embodiment of the present invention;

FIG. 2 is a flowchart showing an imaging control process executed by a controller of an imaging console in FIG. 1;

FIG. 3 is a flowchart showing an abnormal site identification process executed by a controller of a diagnostic console in FIG. 1;

FIG. 4 illustrates a process of identifying the three-dimensional position of an abnormal site;

FIG. 5 shows an example of display of the abnormal site whose three-dimensional position is identified;

FIG. 6 shows an example of display of a register region for the abnormal site; and

FIG. 7 shows another example of display of the abnormal site whose three-dimensional position is identified.

### DETAILED DESCRIPTION OF EMBODIMENTS

Hereinafter, one or more embodiment of the present invention will be described with reference to the drawings. However, the scope of the invention is not limited to the disclosed embodiments.

### [Configuration of Dynamic Image Processing System 100]

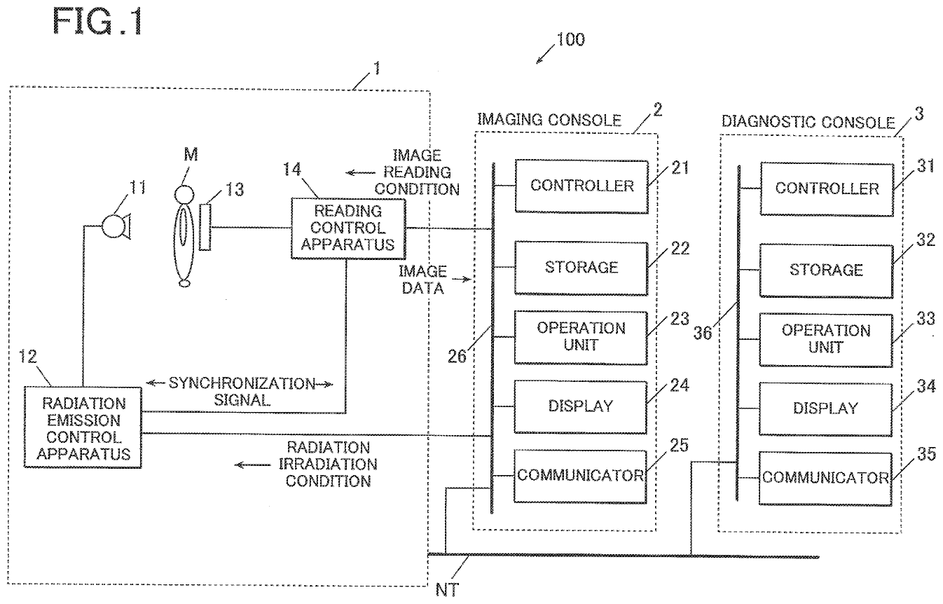

FIG. 1 shows the entire configuration of a dynamic image processing system 100 according to this embodiment.

As shown in FIG. 1, in the dynamic image processing system 100, an imaging apparatus 1 and an imaging console 2 are connected to each other by a communication cable or the like, and the imaging console 2 and a diagnostic console 3 are connected to each other via a communication network NT, such as a LAN (Local Area Network). The apparatuses constituting the dynamic image processing system 100 conform to the DICOM (Digital Image and Communications in Medicine) standard. Communication between the apparatuses is performed in conformity with DICOM.

### [Configuration of Imaging Apparatus 1]

The imaging apparatus 1 is an imager that images the dynamics having a cycle, such as a change of form due to inflation and contraction of lungs accompanying respiratory motion, and heartbeats, for example. The dynamic imaging is to obtain multiple images representing the dynamics of an imaging object by generating pulsed radiation, such as X-rays, and repetitively irradiating the imaging object with the radiation (pulsed irradiation) at predetermined intervals, or by generating radiation with a low dose rate and irradiating the object uninterruptedly and continuously (continuous irradiation). A series of images obtained by dynamic imaging is called a dynamic image. Each of the images constituting the dynamic image is called a frame image. In the following embodiment, a case of dynamically imaging a chest through pulsed irradiation is exemplified and described.

A radiation source 11 is disposed at a position opposite to a radiation detector 13, with an imaging object M intervening therebetween, and irradiates the imaging object M with radiation (X-rays), according to the control by a radiation emission control apparatus 12.

The radiation emission control apparatus 12 is connected to the imaging console 2, and controls the radiation source 11 to perform radiation imaging on the basis of a radiation irradiation condition input through the imaging console 2. The radiation irradiation condition input through the imaging console 2 includes, for example, the pulse rate, the pulse width, the pulse interval, the number of imaging frames per imaging time, the value of X-ray tube current, the value of X-ray tube voltage and an added filter type. The pulse rate is the number of irradiations with radiation per second, and coincides with the frame rate described later. The pulse width is a time period of irradiation with radiation per radiation irradiation. The pulse interval is a time period from the start of one irradiation with radiation to the start of the next irradiation with radiation, and coincides with the frame interval described later.

The radiation detector 13 includes a semiconductor image sensor, such as FPD. The FPD includes, for example, a glass substrate and the like. Multiple detection elements (pixels) are arranged at predetermined positions on the substrate in a matrix manner. The detection elements each detect radiation having been emitted from the radiation source 11 and passed through at least the imaging object M according to the intensity, convert the detected radiation into an electric signal and accumulate the signal. Each pixel includes a switcher, such as TFT (Thin Film Transistor), for example. FPDs include an indirect type that converts X-rays into an electric signal by a photoelectric conversion element through a scintillator, and a direct type that directly converts X-rays into an electric signal. Any of the types may be adopted.

The radiation detector 13 is provided so as to face the radiation source 11, with the imaging object M intervening therebetween.

A reading control apparatus 14 is connected to the imaging console 2. The reading control apparatus 14 controls the switcher of each pixel of the radiation detector 13 to switch reading of the electric signal accumulated in this pixel on the basis of an image reading condition input through the imaging console 2, and reads the electric signal accumulated in the radiation detector 13, thus obtaining image data. This image data is a frame image. The reading control apparatus 14 then outputs the obtained frame image to the imaging console 2. The image reading condition includes, for example, the frame rate, frame interval, pixel size, and image size (matrix size). The frame rate is the number of frame images obtained per second, and coincides with the pulse rate. The frame interval is a time period from the start of one time of an operation of obtaining the frame image to the start of the operation of obtaining the next frame image, and coincides with the pulse interval.

Here, the radiation emission control apparatus 12 and the reading control apparatus 14 are connected to each other, and mutually exchange synchronization signals to synchronize a radiation irradiation operation and an image reading operation with each other.

### [Configuration of Imaging Console 2]

The imaging console 2 outputs the radiation irradiation condition and the image reading condition to the imaging apparatus 1, controls the imaging apparatus 1 to perform the radiation imaging and the operation of reading a radiographic image, and displays the dynamic image obtained by the imaging apparatus 1 for verifying whether or not the image is an image suitable for verifying positioning by an imaging practitioner, such as an imaging technician, and for diagnosis.

As shown in FIG. 1, the imaging console 2 includes a controller 21, a storage 22, an operation unit 23, a display 24, and a communicator 25. These components are connected to each other via a bus 26.

The controller 21 includes a CPU (Central Processing Unit) and an RAM (Random Access Memory). The CPU of the controller 21 reads a system program and various processing programs stored in the storage 22 and deploys the programs in the RAM according to an operation through the operation unit 23, executes various processes including an imaging control process, described later, according to the deployed program, and controls the operation of each component of the imaging console 2 and the radiation irradiation operation and the reading operation of the imaging apparatus 1 in a centralized manner.

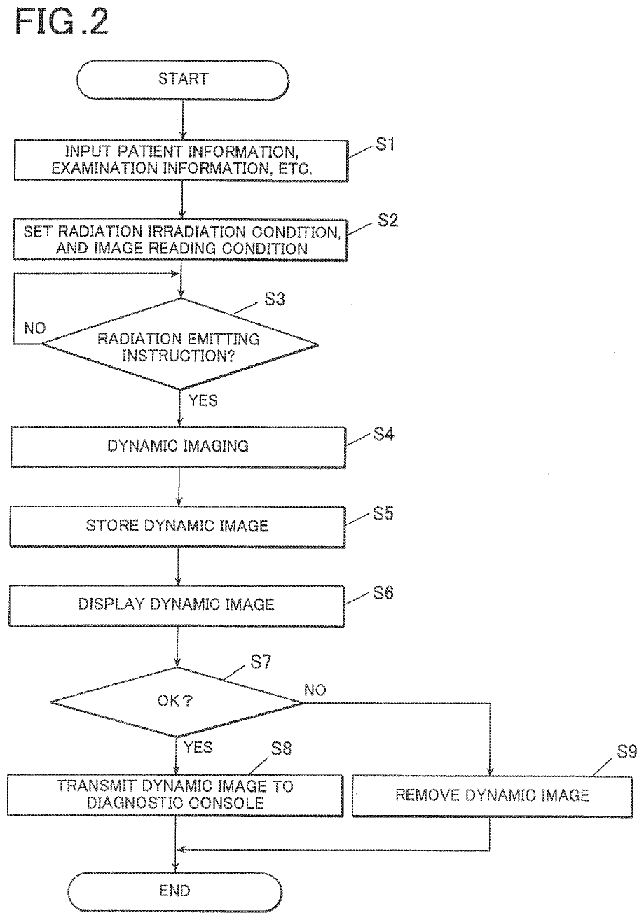

The storage 22 includes a nonvolatile semiconductor memory, or a hard disk. The storage 22 stores various programs to be executed by the controller 21, and parameters required to execute the processes by the programs, or data including processing results. For example, the storage 22 stores programs for executing the imaging control process shown in FIG. 2. The storage 22 stores the radiation irradiation condition and the image reading condition in association with an imaging object site (here, assumed as a chest). The various programs are stored in a form of computer-readable program codes. The controller 21 sequentially executes operations according to the program codes.

The operation unit 23 includes a keyboard provided with cursor keys, numeric input keys and various function keys, and a pointing device, such as a mouse, and outputs, to the controller 21, an instruction signal input through a key operation into the keyboard or a mouse operation. The operation unit 23 may include a touch panel on a display screen of the display 24. In this case, the operation unit 23 outputs, to the controller 21, the instruction signal input through the touch panel.

The display 24 may be a monitor, such as an LCD (Liquid Crystal Display) or a CRT (Cathode Ray Tube), and displays an input instruction, data and the like from the operation unit 23, according to an instruction of a display signal input from the controller 21.

The communicator 25 includes an LAN adaptor, and a modem or a TA (Terminal Adapter), and controls data transmission and reception to and from each apparatus connected to the communication network NT.

### [Configuration of Diagnostic Console 3]

The diagnostic console 3 is a dynamic image processing apparatus for obtaining the dynamic image from the imaging console 2, and displaying the obtained dynamic image and an analysis result of the dynamic image to support a doctor's diagnosis.

As shown in FIG. 1, the diagnostic console 3 includes a controller 31, a storage 32, an operation unit 33, a display 34, and a communicator 35. These components are connected to each other via a bus 36.

The controller 31 includes a CPU and a RAM. The CPU of the controller 31 reads a system program and various processing programs stored in the storage 32 and deploys the programs in the RAM according to an operation through the operation unit 33, executes various processes including an abnormal site identification process, described later, according to the deployed program, and controls the operation of each component of the diagnostic console 3 in a centralized manner

The storage 32 includes a nonvolatile semiconductor memory, or a hard disk. The storage 32 stores various programs including a program for executing the abnormal site identification process in the controller 31, and parameters required to execute the processes by the programs, or data including processing results. These various programs are stored in a form of computer-readable program codes. The controller 31 sequentially executes operations according to the program codes.

The storage 32 stores a previously taken dynamic image in association with patient information (e.g., patient ID, patient name, height, weight, age, gender, etc.), examination information (e.g., examination ID, examination date, imaging object site (chest in this case), the type of dynamics of a diagnosis target (e.g., eupnea, deep breathing, heartbeat, etc.), and the imaging direction (e.g., front, side, oblique position, etc.). Electronic chart information corresponding to the dynamic images may be obtained from an electronic chart apparatus, not shown, and stored in association with the dynamic images.

The operation unit 33 includes a keyboard provided with cursor keys, numeric input keys and various function keys, and a pointing device, such as a mouse, and outputs, to the controller 31, an instruction signal input through a key operation into the keyboard or a mouse operation by a user. The operation unit 33 may include a touch panel on a display screen of the display 34. In this case, the operation unit 33 outputs, to the controller 31, the instruction signal input through the touch panel.

The display 34 may be a monitor, such as an LCD or a CRT, and displays various items according to the instruction of the display signal input from the controller 31.

The communicator 35 includes an LAN adaptor, and a TA or a modem, and controls data transmission and reception to and from each apparatus connected to the communication network NT.

### [Operation of Dynamic Image Processing System 100]

Next, the operation of the dynamic image processing system 100 in this embodiment is described.

### (Operations of Imaging Apparatus 1 and Imaging Console 2)

First, the imaging operations by the imaging apparatus 1 and the imaging console 2 are described.

FIG. 2 shows the imaging control process executed by the controller 21 of the imaging console 2. The imaging control process is executed by cooperation between the controller 21 and the program stored in the storage 22.

First, the operation unit 23 of the imaging console 2 is operated by the imaging practitioner, and the patient information on the subject and the examination information are input (step S1).

Next, the radiation irradiation condition is read from the storage 22 and is set in the radiation emission control apparatus 12, and the image reading condition is read from the storage 22 and is set in the reading control apparatus 14 (step S2).

Next, an instruction of emitting radiation through the operation to the operation unit 23 is waited (step S3). Here, the imaging practitioner positions the imaging object M by disposing this object between the radiation source 11 and the radiation detector 13. Furthermore, an instruction on the respiration state depending on the type of dynamics of a diagnosis target is issued to the subject. When the preparation for imaging is made, the operation unit 23 is operated and a radiation irradiation instruction is input.

When the radiation irradiation instruction is input through the operation unit 23 (step S3; YES), an imaging start instruction is output to the radiation emission control apparatus 12 and the reading control apparatus 14, and the dynamic imaging is started (step S4). That is, the radiation is emitted from the radiation source 11 at a pulse interval set in the radiation emission control apparatus 12, and a frame image is obtained by the radiation detector 13.

After a predetermined number of frames have been taken, an instruction of finishing imaging is output by the controller 21 to the radiation emission control apparatus 12 and the reading control apparatus 14, and the imaging operation is stopped. The number of taken frames is a number allowing at least one respiratory cycle to be imaged.

The frame images obtained by imaging are sequentially input into the imaging console 2, stored in the storage 22 in association with the respective numbers indicating the imaging order (frame numbers) (step S5), and displayed on the display 24 (step S6). The imaging practitioner verifies positioning and the like with reference to the displayed dynamic image, and determines whether the image suitable for diagnosis is obtained by imaging (imaging OK) or reimaging is required (imaging NG). The operation unit 23 is operated to input a determination result.

When a determination result indicating imaging OK is input by a predetermined operation through the operation unit 23 (step S7; YES), each image of the series of frame images obtained by dynamic imaging is assigned information that includes an identification ID for identifying the dynamic image, the patient information, the examination information, the radiation irradiation condition, the image reading condition, the number indicating the imaging order (frame number), and the imaging direction (written in a header region of the image data in the DICOM format, for example), and is transmitted to the diagnostic console 3 via the communicator 25 (step S8). This process is then finished. On the contrary, a determination result indicating imaging NG is input by a predetermined operation through the operation unit 23 (step S7; NO), the series of frame images stored in the storage 22 is removed (step S9), and this process is finished. In this case, reimaging is required.

### [Operation of Diagnostic Console 3]

Next, the operation of the diagnostic console 3 is described.

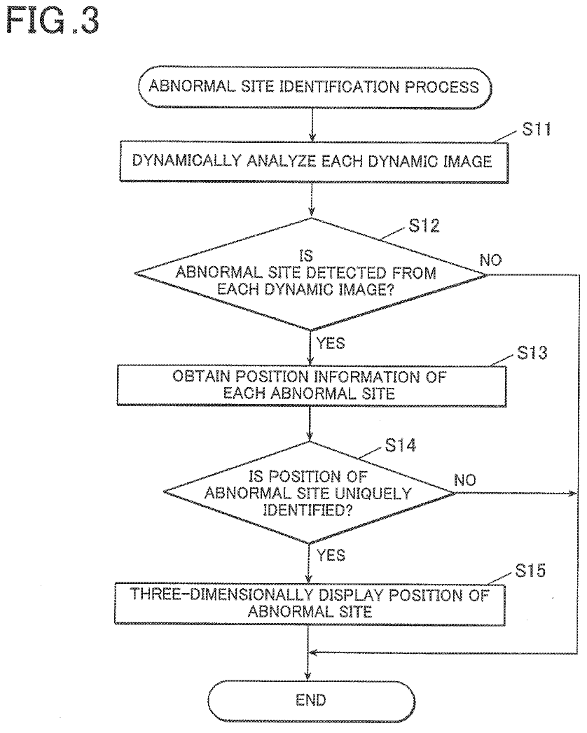

In the diagnostic console 3, when multiple dynamic images related to one subject (dynamic images whose types of dynamics are the same but whose imaging directions are different from each other) are received from the imaging console 2, the abnormal site identification process shown in FIG. 3 is executed by cooperation between the controller 31 and the program stored in the storage 32. Note that the following description is made with an example of a case where dynamic images taken from the front and a side (left side) are received as the multiple dynamic images related to the one subject.

In the abnormal site identification process, first, dynamic analysis is performed for each of the received dynamic images (step S11). Here, the dynamic analysis may be ventilatory analysis, or blood flow analysis. Note that when the dynamic analysis is performed, an attenuation process for various structures (e.g., costae attenuation process), an enhancement process (e.g., blood vessels enhancement process) or the like may be performed, as required. When the dynamic analysis is performed, adjustment may be made by increasing or reducing the number of frame images of each dynamic image, in order to increase the ratio of frame images where the respiratory movement timings coincide with each other in the dynamic images. When the number of frame images is adjusted, a pixel value interpolation process may be performed on the basis of previous and subsequent frame images, as required.

Next, as a result of the dynamic analysis, it is determined whether an abnormal site is detected or not (step S12). Here, the abnormal site is detected from the gradation of the signal value of each pixel of each of the frame images constituting the dynamic image. For example, a site where the signal values are less than a predetermined threshold are regarded as the abnormal site. Note that the abnormal site may be detected from, for example, the amount of variation, variation speed, variation timing, etc. of the signal values. For example, a region where the tendency of the variation of the signal values is different from surrounding areas is detected as the abnormal site. Alternatively, the abnormal site may be detected from the amount of movement, moving velocity, movement timing and the like of the structure displayed in the dynamic image. Alternatively, a site that falls into a predetermined pattern may be detected as the abnormal site through a pattern matching process. A site where the size (e.g., the diameter, length, etc.) of a structure displayed in the dynamic image is less than a predetermined threshold may be detected as the abnormal site.

If it is determined that any abnormal site is not detected from each dynamic image in step S12 (step S12; NO), the abnormal site identification process is finished.

On the contrary, if it is determined that the abnormal site is detected from each dynamic image in step S12 (step S12; YES), the position information on each abnormal site is obtained (step S13).

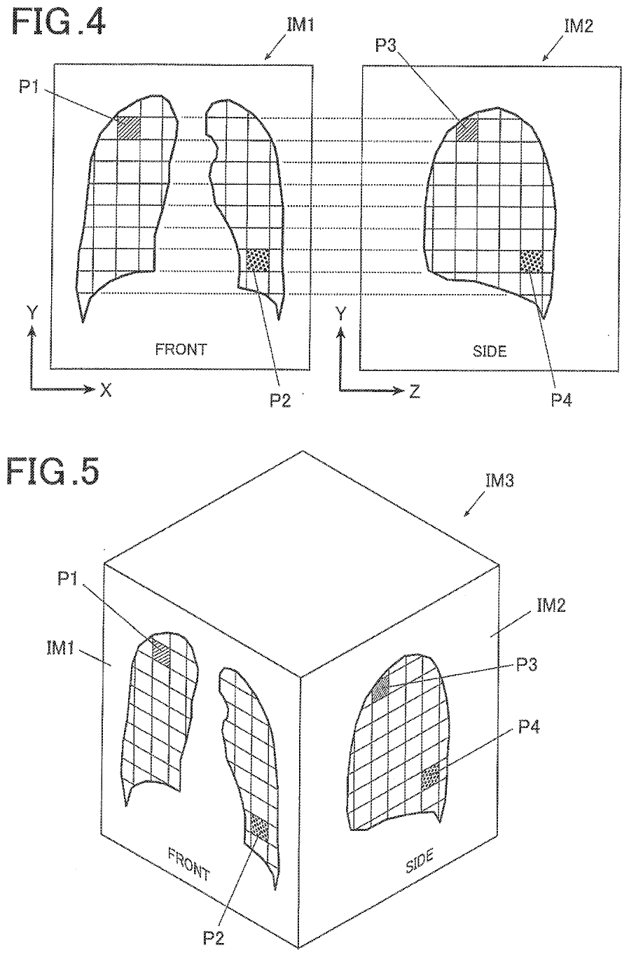

For example, as shown in FIG. 4, if abnormal sites P1 and P2 are detected as a result of the dynamic analysis for the dynamic image taken from the front (hereinafter called a front dynamic image), position information indicating the positions (coordinates) of the abnormal sites P1 and P2 in the lateral direction (X direction) and vertical direction (Y direction) is obtained. If abnormal sites P3 and P4 are detected as a result of the dynamic analysis for the dynamic image taken from a side (left side) (hereinafter called a side dynamic image), position information indicating the positions (coordinates) of the abnormal sites P3 and P4 in the vertical direction (Y direction) and anteroposterior direction (Z direction) is obtained. Here, the image shown on the left of FIG. 4 is a front analysis result image IM1 that represents the dynamic analysis result of the front dynamic image. The image shown on the right of this diagram is a side analysis result image IM2 that represents the dynamic analysis result of the side dynamic image. Each of the analysis result images IM1 and IM2 is a gradation image that represents the result of dynamic analysis in gradations on a pixel-by-pixel basis. In the analysis result images IM1 and IM2, the detected abnormal sites P1 to P4 are displayed in colors different from those of normal sites.

Next, based on the position coordinates of each abnormal site obtained in step S13, it is determined whether the position (three-dimensional position) of the abnormal site is uniquely determined or not (step S14).

Specifically, if the abnormal site detected in the front dynamic image and the abnormal site detected in the side dynamic image coincide with each other in the vertical direction (Y direction), the position (three-dimensional position) of the abnormal site is assumed to be uniquely determined. Note that, for example, if multiple abnormal sites coinciding in the vertical direction (Y direction) with the abnormal site detected in the front dynamic image are detected in the side dynamic image, the size of the abnormal site detected in the front dynamic image is considered and the site may be determined to be coincide in the vertical direction (Y direction) with the abnormal site having the same size as the aforementioned abnormal site has.

On the other hand, if the abnormal site detected in the front dynamic image and the abnormal site detected in the side dynamic image do not coincide with each other in the vertical direction (Y direction), the positions (three-dimensional positions) of the abnormal sites are assumed not to be uniquely determined. Note that if the positions of abnormal sites are not uniquely determined, for example, it may be determined whether an abnormal site is detected also in another frame image of the side dynamic image or not, and when the abnormal site is detected also in the other frame image, it may be determined whether the positions (three-dimensional positions) of this abnormal site and the abnormal site detected in the front dynamic image may be uniquely determined or not. If the position of abnormal site is not uniquely determined, a dynamic image taken in another direction may be reobtained (received again) from the imaging console 2 and it may be determined whether or not the position (three-dimensional position) of the abnormal site is uniquely determined with respect to the abnormal site detected again from this dynamic image.

For example, in the example shown in FIG. 4, the abnormal site P1 detected in the front dynamic image and the abnormal site P3 detected in the side dynamic image coincide in the vertical direction (Y direction) with each other. It is determined that the position (three-dimensional positions) of the abnormal site is uniquely identified with respect to the abnormal site P1 and the abnormal site P3. The abnormal site P2 detected in the front dynamic image and the abnormal site P4 detected in the side dynamic image coincide in the vertical direction (Y direction) with each other. It is determined that the position (three-dimensional position) of the abnormal site is uniquely identified with respect to the abnormal site P2 and the abnormal site P4.

If it is determined that the position of the abnormal site is not uniquely determined in step S14 (step S14; NO), the abnormal site identification process is finished.

On the contrary, if it is determined that the position of the abnormal site is uniquely determined in step S14 (step S14; YES), the position of the abnormal site is three-dimensionally displayed (step S15) and the abnormal site identification process is finished.

Specifically, for example, as shown in FIG. 4, if the position (three-dimensional position) of the abnormal site is uniquely determined on the basis of the abnormal site P1 and the abnormal site P3 and the position (three-dimensional position) of the abnormal site is uniquely determined on the basis of the abnormal site P2 and the abnormal site P4, the abnormal sites P1 to P4 are three-dimensionally displayed on a three-dimensional image IM3 where the front analysis result image IM1 and the side analysis result image IM2 are three-dimensionally displayed, as shown in FIG. 5. Accordingly, when a doctor views the three-dimensional image IM3, he/she can easily grasp that the abnormal site P1 (=P3) detected at an upper part of the right lung resides at an anterior part of the lung, and the abnormal site P2 (=P4) detected at a lower part of the left lung resides at a posterior part of the lung.

As described above, in this embodiment, an abnormal site of an imaging object from a radiographically taken dynamic image is detected, and a three-dimensional position of the abnormal site is identified from a positional relationship between the abnormal site of the imaging object detected from one dynamic image and the abnormal site of the imaging object detected from another dynamic image when the abnormal site is detected from a plurality of dynamic images radiographically taken in a plurality of directions. The series of processes including detection of the abnormal site to identification of the three-dimensional position of the abnormal site can be easily performed.

Furthermore, in this embodiment, the abnormal site is detected on the basis of the dynamic analysis result of the dynamic image. Consequently, the series of processes including detection of the abnormal site related to an invisible function, such as ventilation or blood flows, to identification of the three-dimensional position of the abnormal site can be easily performed.

In this embodiment, the detected abnormal site is displayed on the display 34. Accordingly, the detected abnormal site can be easily grasped.

In this embodiment, when the three-dimensional position of the abnormal site is detected, the abnormal sites (e.g., abnormal sites P1 to P4) are displayed on the three-dimensional image IM3 that three-dimensionally represents the analysis result image (e.g., the analysis result image IM1) indicating the dynamic analysis result of one dynamic image and the analysis result image (e.g., the analysis result image IM2) indicating the dynamic analysis result of another dynamic image. Consequently, the three-dimensional position of the abnormal site can be easily and correctly grasped.

The embodiments of the present invention have thus been described above. However, the description in the above embodiments is preferable examples of the present invention, which is not limited thereto.

For example, in the above embodiment, the case where the imaging object site is the chest has been exemplified and described. Alternatively, the present invention is also applicable to a case where the analysis process is performed for a dynamic image obtained by imaging another site.

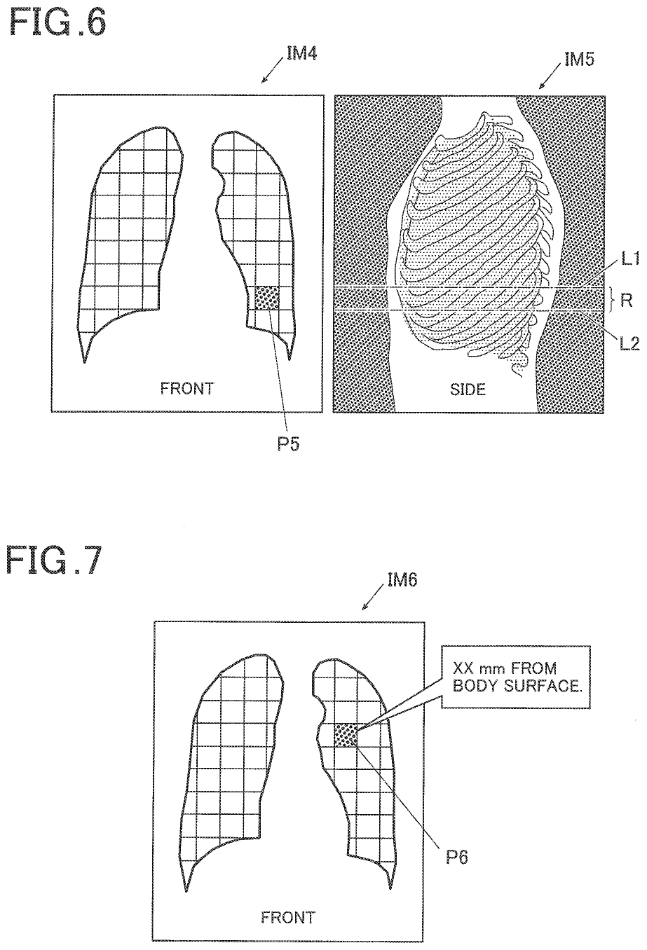

In the above embodiment, the case where the front dynamic image and the side dynamic image related to one subject are received has been exemplified, and the abnormal site identification process (see FIG. 3) has been described. For example, when the front dynamic image and a side still image that are related to one subject are received, the dynamic analysis may be performed only for the front dynamic image. When the abnormal site is detected from the front dynamic image, an analysis result image IM4 of the front dynamic image indicating an abnormal site P5, and the side still image IM5 may be associated with each other and displayed on the display 34, and a first line L1 indicating the upper end position of the abnormal site P5 and a second line L2 indicating the bottom end position may be displayed on the still image IM5, as shown in FIG. 6. A region (register region) that is sectioned by the first line L1 and the second line L2 and has a width R is displayed on the still image IM5, thereby enabling a region having a high possibility of presence of the abnormal site P5 to be indicated. Accordingly, the efficiency of diagnosis using the still image IM5 can be improved. Note that the front dynamic image, where the abnormal site is detected, is not limited to that taken at the same time of the side still image. Alternatively, the image may be taken previously.

In the above embodiment, in the abnormal site identification process, if the position of the abnormal site is determined to be uniquely identified (step S14; YES), the position of the abnormal site is three-dimensionally displayed (step S15), as shown in FIG. 5. Alternatively, for example, as shown in FIG. 7, only an analysis result image IM6 of a front dynamic image indicating an abnormal site P6 may be displayed on the display 34. When an input designating the abnormal site P6 through a predetermined operation on the operation unit 33 is made, information indicating the depth in the depth direction (Z direction) and the size of the abnormal site P6 (for example, “XX mm from body surface.” or “Size is XX mm,” etc.) may be pop-up displayed.

In the above embodiment, as shown in FIG. 5, when the position of the abnormal site is three-dimensionally displayed, not only the three-dimensional display but also the position of the abnormal site may be displayed in text (e.g., Right lung (upper part, intermediate part, lower part), Left lung (upper part, intermediate part, lower part), etc.).

For example, if a chest anatomic information data set is stored in the storage 32, the position of the abnormal site may be displayed in text (e.g., upper lobe, intermediate lobe, and inferior lobe) based on the anatomic information data set.

In the above embodiment, based on the position information on all the pixels of each image among the front dynamic image and the side dynamic image, voxel data (three-dimensional data) may be generated.

In the above embodiment, the front dynamic image and the side dynamic image used for the abnormal site identification process may be sequentially taken dynamic images, or simultaneously taken dynamic images.

For example, the above description discloses an example using the hard disk, the semiconductor nonvolatile memory or the like as a computer-readable medium storing the program according to the present invention. However, the scope is not limited to this example. A portable recording medium, such as CD-ROM, may be adopted as another computer-readable medium. A medium for providing, via a communication line, data in the computer-readable recording medium storing the program according to the present invention may be carrier waves.

Alternatively, the detailed configuration and detailed operation of each of apparatuses constituting the dynamic image analysis system may also be appropriately modified in a scope without departing from the spirit of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-122598, filed on 28st of June, 2018, is incorporated herein by reference in its entirety.

What is claimed is:

1. A dynamic image processing apparatus, comprising a hardware processor that detects an abnormal site of an imaging object from a radiographically taken dynamic image, and identifies a three-dimensional position of the abnormal site from a positional relationship between the abnormal site of the imaging object detected from one dynamic image and the abnormal site of the imaging object detected from another dynamic image when the abnormal site is detected from a plurality of dynamic images radiographically taken in a plurality of directions.

2. The dynamic image processing apparatus according to claim 1, wherein the hardware processor detects the abnormal site, based on a dynamic analysis result of the dynamic image.

3. The dynamic image processing apparatus according to claim 2, wherein the hardware processor displays the detected abnormal site on a display.

4. The dynamic image processing apparatus according to claim 3, wherein when the three-dimensional position of the abnormal site is identified, the hardware processor displays the abnormal site on a three-dimensional image that three-dimensionally represents an analysis result image indicating a dynamic analysis result of the one dynamic image and an analysis result image indicating a dynamic analysis result of the other dynamic image.

5. The dynamic image processing apparatus according to claim 1, wherein the hardware processor detects the abnormal site, based on an amount of movement or an amount of deformation of a structure displayed in the dynamic image.

6. The dynamic image processing apparatus according to claim 3, wherein the hardware processor displays the detected abnormal site on the display in association with an image radiographically taken in a direction different from an imaging direction of the dynamic image in which the abnormal site is detected, and displays, on the image, a register region for the abnormal site, when displaying the detected abnormal site on the display.

7. A computer-readable recording medium storing a program for causing a computer to function as
a hardware processor that detects an abnormal site of an imaging object from a radiographically taken dynamic image, and identifies a three-dimensional position of the abnormal site from a positional relationship between the abnormal site of the imaging object detected from one dynamic image and the abnormal site of the imaging object detected from another dynamic image when the abnormal site is detected from a plurality of dynamic images radiographically taken in a plurality of directions.
